(19) United States
(12) Patent Application Publication
(10) Pub. No.: US 2003/0159693 A1
(43) Pub. Date: Aug. 28, 2003

(54) Breath-activated, microprocessor controlled system for pulmonary drug delivery

(76) Inventors: Richard Melker, Gainesville, FL (US); Johannes H. Van Oostrom, Gainesville, FL (US)

Correspondence Address: VAN DYKE & ASSOCIATES, P.A., 7200 LAKE ELLENOR DRIVE, SUITE 252, ORLANDO, FL, 32809, US

(21) Appl. No.: 10/087,894

(22) Filed: Feb. 27, 2002

(51) Int. Cl. A61M 16/00; A61M 11/00

(52) U.S. Cl. 128/203.12; 128/200.23; 128/204.22

(57) ABSTRACT

A small breath-activated, microprocessor controlled device that can be used with any drug formulation appropriate for pulmonary delivery is disclosed. A fixed-resistor housing incorporates a microprocessor and pressure transducer to detect pressure changes associated with patient derived airflow across the device. The microprocessor determines the flow rate and tidal volume over several breaths and averages them. Depending on where in the brocho-pulmonary tree the drug is to be administered, the microprocessor will precisely meter the drug in the appropriate phase of the respiratory cycle and deliver small aliquots of the formulation over several breaths until the total dose is administered.

### FIELD OF THE INVENTION

The present invention relates in general to systems for delivery of drug formulations, and particularly to a breath-activated, microprocessor controlled device for intrapulmonary delivery of drug formulations.

### BACKGROUND OF THE INVENTION

Intrapulmonary drug delivery techniques are used to treat a variety of diseases, such as asthma, chronic obstructive pulmonary disease (COPD) such as chronic bronchitis and emphysema, and other lung diseases characterized by obstruction to airways and shortness of breath. Of these diseases, asthma is perhaps the most pervasive and troubling. Approximately 17 million Americans suffer from asthma (CDC Forcasted State-Specific Estimates of Self-Reported Asthma Prevalence-United States, December 1998 MMWR; 47(47):1022-1025) The incidence of death attributable to this disease has increased by nearly sixty percent over the last two decades, and currently accounts for more than 5,000 deaths per year. (Centers for Disease Control and Prevention, CDC Surveillance Summaries, MMWR; 47(SS-1), Apr. 24, 1998) Direct and indirect medical expenditures associated with asthma are estimated at approximately $13 billion annually. (Weiss, K. B., et al. An Economic Evaluation of Asthma in the U.S. New England Journal of Medicine 326:862-6, 1992) Numerous intrapulmonary drug delivery techniques have been developed to treat this and other respiratory disorders, with varying levels of success.

Metered dose inhalers (MDIs), have emerged as the preferred delivery system for inhaled medications for asthmatics, because of their ability to deliver a drug to a specified site within the broncho-pulmonary tree, high degree of patient acceptance, portability and multiple dose capacity. (Clark A R. Aerosol Sci Tech 1995; 22:374-91) The most common MDIs used for out-patient treatment of asthma, as well as other respiratory disorders include the press-and-breathe systems, the breath-actuated systems, and dry-powder inhalers. Typically, these MDIs comprise a housing in communication with a canister containing an active drug, a surfactant and a propellant. One of two types of medicines: an anti-inflammatory medication, such as, for example cromolyn, or a bronchodilator medication, such as, for example beta2-agonist, are routinely used. Nearly all asthma sufferers depend on MDI's for disease control and symptomatic relief. However, despite almost universal use of MDI's, it is estimated that greater than 50% of patients are unable to use these devices efficiently because of the varying levels of skill required to operate each type of MDI. (Levit, M. A., et al. Annals of Emergency Medicine, September 1995, 26(3), 273-277).

Typically, a patient must coordinate activation of a canister containing a drug formulation, with a specific phase of an inspiratory cycle, to enable adequate delivery of the medicament deep into the lungs. (Kemp, J. et. al. 1997 Annals of Allergy, Asthma & Immunology 79: 322-326) Coordination of the specific sequence of steps may be difficult to achieve for adult and child alike. (NIH Pub. No. 97-44051, 1-154). Common errors include: improper activation during the wrong stage of inspiratory flow, inadequate breath holding, or multiple activations of the aerosol during inspiration. (Boccuti L., et al. Annals of Allergy, Asthma and Immunology September 1996, 77(3), 217-221; NIH Pub. No. 97-44051, 1-154). Further, young children or the elderly may have insufficient hand strength to apply sufficient manual pressure to the top and bottom of the device to activate it. (Gray, S. L. et al. 1996. Archives of Internal Medicine 156(9): 984-988) These user errors reduce the effectiveness of each system and decrease the delivery of the medication to the site of need.

Another problem with traditional MDIs is their relative inefficiency. Significant loss of drug dosage associated with oropharyngeal deposition often occurs with improper use of the MDI. Deposition in the oral cavity can lead to irritation, foul taste and could predispose the patient to oral candidiasis or “thrush” which may cause the patient to avoid using the medication. Part of the reason for this is the fact that aerosolized particles are often not within a respirable range (i.e. approximately 1-5 microns) immediately upon release form the canister. Thus, unlike most conventional drug products, particle size distribution of the delivered dose is critical for proper absorption of inhalation aerosols. Particle size is dependent on the particular formulation, mouthpiece, and the valve.

One method to increase the delivery of medicine to the lungs is to slow the aerosolized particles released from the canister long enough to allow for partial evaporation. Spacers are commonly affixed to MDI's to decrease oropharyngeal deposition and reduce potential systemic absorption of inhaled medicaments by increasing the distance the formulation must travel before reaching the lungs. This extra distance traveled provides added time for the drug to evaporate to a respirable size prior to entering a patient. Spacer may also be used as holding chambers when the coordination between the firing of the MDI and the onset of the inspiration cannot be achieved simultaneously. Approximately 80% of the dose from an MDI is swallowed when a spacer is not used. (NIH Pub. No.97-44051, 1-154). For inexpensive drugs this is not a problem, especially if there is a wide safety profile. However, for drugs with narrow safety profiles and high costs, or where precise delivery is needed, the available MDI's have been problematic. Further, spacers do not help at all when a patient has inadequate breathing.

In order to overcome some of the problems associated with poor MDI design and/or technique, aerosol breath-activated inhalers were developed. These devices are more cost-effective than press and release MDIs because they release a dose of a medicament in response to inhalation from the patient. (Langley, PC. 1999 Clinical Therapeutics 21, 236-253) The majority of currently available breath activated MDIs, such as the Clickhaler®, Accuhaler®, Turbohaler®, Rotahaler®, Diskhaler®, Spinhaler®, and Autohaler® require a user to employ a closed mouth technique, wherein the user places the mouthpiece between the lips, sucks in steadily and deeply, and holds his or her breathe for approximately 10 seconds. This generates an air-stream which actuates the device to release an all-or none dose of medicine for pulmonary delivery.

Most breath-activated inhalers utilize the energy in the patient's inspiration as the power source for activating the device. However, these devices are not particularly useful when the patient generates very low airflow rates. For a variety of reasons, such as, for example, during an acute attack of asthma, a patient's inspiratory flow may be insufficient to actuate the device when medication is most needed. (Newman, S. Journal of Pharmaceutical Science September 1996:85(9), 960-964) Also, since both dose and particle size distribution is dependent on the flow characteristics of an air-stream generated by a user, a low inspiratory flow-rate, may improperly activate the MDI and provide less than the prescribed dose.

U.S. Pat. No. 6,260,549 (Clavius Devices, Inc.) discloses a breath-activated MDI (sold commercially as the Easi-Breathe® Inhaler) that permits an open-mouth inhalation technique, whereby the MDI is activated through inhalation while the MDI is in proximity, but not in direct contact with the patient's mouth. This technique has been shown to lead to enhanced drug delivery to the lung compared with the more conventional closed-mouth technique. (NIH Publication No. 97-44051, 1-154). The '549 patent includes an enhanced sensor to allow activation of the device and delivery of medicine, even when a patient generates very low inspiratory flow rates. However, one significant disadvantage of the '549 device is that it releases the entire dose of medicine in response to a signal from the sensor. Thus, as with other devices, which release the medicine all at once, a certain portion of the medicine will not evaporate to within a respirable range prior to assimilation by a patient, and therefore will not reach its target. Further, because the entire dose is delivered, the patient must still hold their breath for at least 10 seconds to allow the entire dose to penetrate pulmonary circulation. This may prove difficult for those patients experiencing an acute attack and therefore unable to hold their breath. Thus, the ideal MDI would permit incremental release of portions of an entire dose over several breaths to ensure that the maximum quantity of the medicament is evaporated to a respirable range prior to entry into a patient. None of the currently available breath-activated inhalers average the flow rate and tidal volume of a patient's inspiratory cycle, and then meter the dose over several breaths. Unfortunately, despite a greater understanding of the problems and solutions to optimal intrapulmonary delivery, the goal has not been realized. Numerous approaches to improving delivery to the lungs have been attempted, but several hurdles remain before optimal lung delivery can be achieved. This invention introduces a new approach to the problem of precise delivery of the appropriate concentration of a drug to the appropriate portion of the broncho-pulmonary tree.

### SUMMARY OF THE INVENTION

Disclosed herein is a small breath-activated, microprocessor controlled device for use with any drug formulation appropriate for pulmonary delivery. The device consists of a fixed resistor housing into which a canister or other delivery system containing a drug formulation can be introduced. Since the resistance of the housing is fixed and known, flow through the device is proportional to the pressure drop across the device. The system incorporates a pressure transducer capable of detecting sub-atmospheric pressure drop across the device resulting from patient inhalation. The pressure drops are converted to electrical signals that are sent to a microprocessor, which then calculates the average flow rate and tidal volume over several breaths. The housing includes a valve or triggering means activated by the microprocessor to precisely deliver known quantities of the formulation into the airflow stream during the appropriate phase of the respiratory cycle. By delivering a precise concentration of medication to the appropriate lung target the present system will reduce waste associated with oropharyangael deposition or swallowing, and provide better therapeutic results. Further, use of a fixed resistor system in lieu of an expensive flow meter, reduces the average cost of production, thereby making the device available to a wider number of users.

Accordingly, it is an object of the present invention to provide a breath-activated, microprocessor controlled apparatus for pulmonary delivery of a drug formulation.

It is a further object of the present invention to provide a breath-activated, microprocessor controlled apparatus for pulmonary delivery of a medication using an open-mouth technique position.

It is a further object of the present invention to provide a breath-activated microprocessor controlled, apparatus for pulmonary delivery of a medication using a closed-mouth technique position.

It is a further object of the present invention to provide a breath-activated, microprocessor controlled apparatus for pulmonary delivery of air-mixed medication that provides a programmable microprocessor.

It is a further object of the present invention to provide a breath-activated, microprocessor controlled apparatus for pulmonary delivery of air-mixed medication, which incorporates a programmable microprocessor capable of detecting different medicaments.

It is a further object of the present invention to provide a breath-activated microprocessor-controlled device capable of measuring flow rate and tidal volume over several breaths and producing an average therefrom.

It is a further object of the present invention to provide a breath-activated, microprocessor controlled apparatus capable of incremental delivery of a medication over several breaths until the entire dose is delivered.

It is a further object of the present invention to provide a breath-activated, microprocessor controlled apparatus capable of delivering aliquots of a medication during the appropriate phase of a respiratory cycle to reach a desired target.

It is yet a further object of the present invention to provide a breath-activated microprocessor controlled apparatus capable of monitoring multiple breaths and calculating the dose of drug required for delivery during subsequent breaths.

It is a further object of the present invention to provide a breath-activated, microprocessor controlled apparatus for pulmonary delivery of air-mixed medication that dispenses liquid medication.

It is a further object of the present invention to provide a breath-activated microprocessor controlled, apparatus for pulmonary delivery of air-mixed medication that dispenses dry medication.

It is still a further object of the present invention to provide a breath-activated microprocessor controlled, apparatus for pulmonary delivery of air-mixed medication that is enabled to record the number of doses dispensed from a canister containing medication as well as the number of doses remaining in the canister.

It is a further object of the present invention to provide a breath-activated, microprocessor controlled apparatus for pulmonary delivery of air-mixed medication that does not require patient interaction.

It is a further object of the present invention to provide a breath-activated microprocessor controlled, apparatus for pulmonary delivery of air-mixed medication that may be manually activated.

It is yet a further object of the present invention to provide a breath-activated, microprocessor controlled apparatus for pulmonary delivery of air-mixed medication that provides an electromechanical discharge of a medicament and uses a battery incorporated into the housing as the power supply.

These and other objects of the present invention will be apparent to those of ordinary skill after review of the specification and claims in view of the figures.

### BRIEF DESCRIPTION OF THE FIGURES

FIG. 1 shows a schematic drawing of the present invention showing all components in place for use.

FIG. 2 shows a flow algorithm of the present invention.

### DETAILED DESCRIPTION OF THE INVENTION

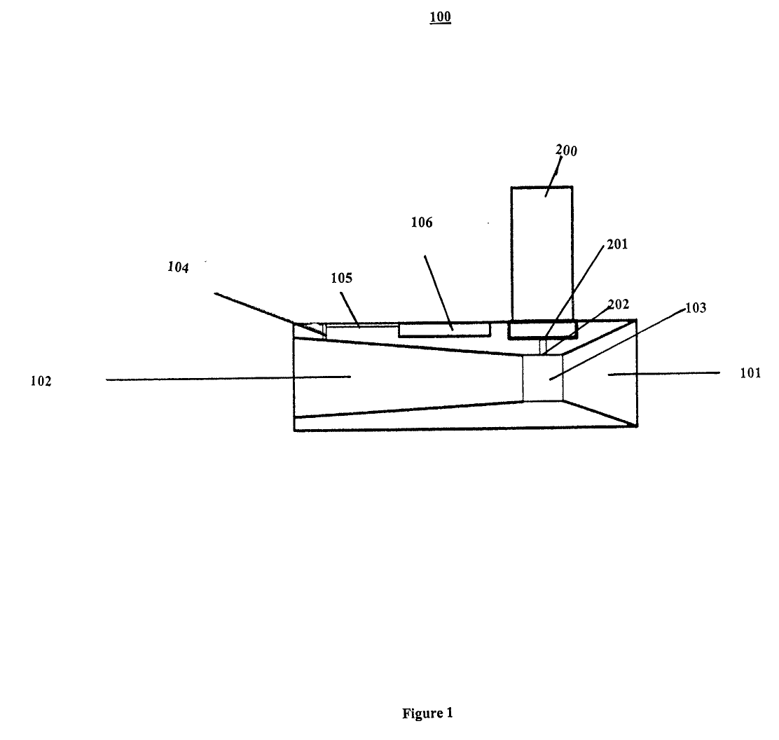

FIG. 1 shows the apparatus generally depicted at 100 as it would be used by a patient. The device has a proximal end 101 and distal end 102. The proximal end has a fixed resistor 103 integral with the apparatus 100. Unlike most presently available devices, the patient will not be required to interact with the device to receive a dose of a medicament. In use a patient will place the distal end 102 of the device in his or her mouth and breath normally. A pressure transducer tap 104 and pressure transducer 105 located in the distal end 102 detects sub-atmospheric pressure drops as air moves through the apparatus 100 and then sends a signal to a microprocessor 106 that will determine the flow rate and tidal volume over several breaths and average them. A user may also use the open mouth technique to achieve similar results. Because the pressure transducer measures sub-atmospheric pressure drops across the device, patients with very low inspiratory flow will be capable of activating the device.

A canister 200 containing some combination of a drug, excipient and/or surfactant appropriate for pulmonary delivery, is inserted into the apparatus 100. The drug used in the canisters may be any drug appropriate for intrapulmonary delivery, such as for example, bronchodilators, steroids, mast-cell stabilizers, anti-inflammatories, or any other drug capable of entering pulmonary or systemic circulation in the body following inhalation, such as insulin, peptides, growth hormones and the like.

The container is held in place via a rubber ring 201 that ensures leak-proof sealing of the container. A valve 202 on the canister provides a route to dispense an appropriate dose from the canister upon activation. Once the drug is dispensed it is mixed with air prior to entering a patient.

Depending on where in the broncho-pulmonary tree the drug is to be deposited, the microprocessor 105 will precisely meter the drug in the appropriate phase of the respiratory cycle. The microprocessor 105 will continue to direct delivery of small aliquots of medication over several breaths until the total dose is delivered. Throughout the delivery cycle, the pressure transducer 105 senses changes in pressure of air flowing across the apparatus and sends an appropriate signal to the microprocessor 105. The microprocessor 105 then evaluates each signal and based on flow and tidal volume will calculate how much drug needs to be delivered during subsequent breaths. The device is extremely small and is preferably designed to be handheld. The device is designed for use with liquid or dry medicaments, as well as any other drug formulation capable of being administered with an MDI. Preferably the device is electrically activated, but it may also be designed with electromechanical elements. Electrical circuitry, electromechanical components, and modes of canister actuation that may be utilized in the present invention are similar to those described in U.S. Pat. No. 6,260,549, which is incorporated herein by reference to the extent that it is not inconsistent with the present teachings. Those skilled in the art will appreciate that the method of actuation taught in the '549 patent may be implemented for use, and incorporated into, the subject apparatus.

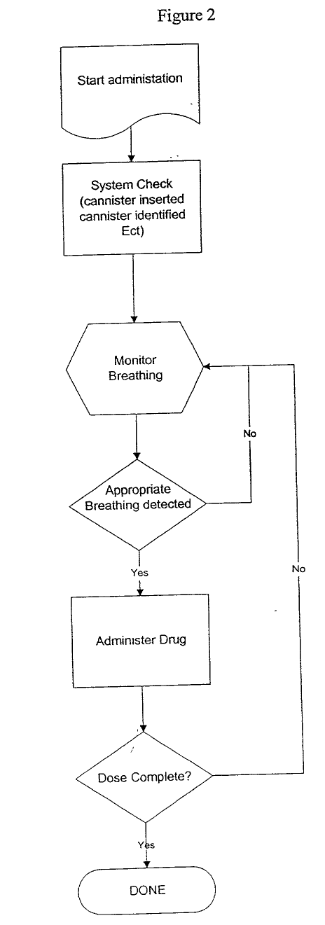

FIG. 2 is a flow algorithm of this system. In use, the subject would place the device in his or her mouth and breath normally. The system then evaluates multiple parameters to insure that the canister is properly situated, and the contents of canister are known. The microprocessor then monitors patient breathing over several breaths to calculate an average flow rate and tidal volume. Without input from the subject, the apparatus properly meters the dose over several additional breaths to insure maximum efficiency. By evaluating the parameters of each breath, the microprocessor calculates whether the medicament was appropriately given, factoring in the correct timing of the inspiratory cycle, correct flow rate and how much drug remains to be given. The medicament then continues to be metered over several breaths until the total dose is given. For delivery deep in the lung (i.e. to the alveoli) the drug is delivered very early in the inspiratory cycle only if a sufficiently low flow rate is sensed. For delivery higher in the brocho-pulmonary tree, the drug is delivered later in the inspiratory cycle and higher flow rate would be permissible.

In a typical cycle of use, a canister containing the drug under pressure is inserted into the device. A system for identifying the medication can be included in the canister and read by the microprocessor in the device or alternatively the microprocessor can be programmed for the drug by connecting it to a computer in the physician's office or at a pharmacy or over the internet. By entering additional patient information such as body weight or height (body length) the microprocessor can calculate the dose of drug to be given and the flow rate that should be generated during quiet breathing. Additionally, the microprocessor can be programmed to deliver the drug during the appropriate time in the inspiratory cycle to deposit the drug in the appropriate location in the broncho-pulmonary tree. The device may also be designed to permit manual activation in the event electrical activation fails. Further, the device may be equipped with an auditory, visual or vibratory indicator, or combination thereof, that provides feedback to a patient if breathing is too high or too low. This added safety measure allows a user to quickly assess whether the medication has been administered adequately.

The housing preferably has a fixed resistor. Flow through the device is therefore proportional to the drop in pressure across the device that occurs when a patient inhales. The system incorporates a pressure transducer capable of detecting sub-atmospheric pressure drops across the device and converting the drop in pressure to electrical signals. An alternate embodiment of the device may incorporate movable parts such that their movement from one location to another within the housing changes the resistance to air flow through the housing. This ability to change the resistance to air flow could aid small children or patients having extremely shallow inhalations. Alternatively the pressure transducer can be made to have sufficient sensitivity to detect the inhalation flow and actuate the device even when very small volumes of air flow through the device. One knowledgable in the field will recognize that the present device could also incorporate flow meter, such as, for example a flow-sensing resistor, a thermally sensitive crystal a mechanical vane and switch, and others that are known in the art.

Since canisters have a known quantity of medication and the dose administered is also known, the present device may also utilize a dose counter to indicate to a patient how much of a medicament remains in a canister. By providing a patient with such information common situations where patients run out of medicine will be minimized. The device may also be enabled to record the time a dose was given and may also include an audible warning to prompt the user when to take the next dose.

One significant advantage offered by the present device is more precise delivery of medication by the intrapulmonary route than any existing MDI device. This reduces waste of expensive medication, ensures safe administration of the drug for those drugs with a low margin of safety, and permits application at a specific site in the broncho-pulmonary tree is desired. Hence, by delivering the appropriate drug, in the appropriate dose, to the appropriate site, waste is reduced resulting in lower overall costs to a patient. Also, use of the present device reduces side effects associated with improper use, leading to greater patient compliance with a prescribed therapeutic regime. Another advantage of the present device an improved delivery system, which accommodates existing and new drugs currently being developed. Thus, canisters containing different drugs may be used, without the need to modify the apparatus, thereby reducing user costs over time. Further, since the device uses a fixed resistor in lieu of a costly flow meter, the present device will be accessible to a larger number of patients than currently available MDIs.

One knowledgeable in the field will recognize that the various embodiments of the disclosed MDI may be used in combination with a wide variety of drugs appropriate for pulmonary delivery, and use of the present device is not limited to the treatment of the specific disease or conditions described herein.

The teachings of the references cited throughout the specification are incorporated herein by this reference to the extent they are not inconsistent with the teachings herein.

We claim:

1. A breath-activated, microprocessor controlled apparatus for pulmonary delivery of air-mixed medication comprising:
a). a housing having a proximal end comprising an inflow port with a fixed resistor in communication with ambient air, and a distal end comprising a mouthpiece, wherein said mouthpiece is in communication with said inflow to permit air to flow through said housing;
b). a pressure transducer for producing a signal in response to changes in airflow through said housing;
c). a programmable microprocessor;
d). a replaceable canister containing a medicament;
e). electrical circuitry, wherein said circuitry permits communication between said pressure transducer, said microprocessor, and said canister; and

2. The apparatus of claim 1 wherein said apparatus is capable of delivering liquid medication.

3. The apparatus of claim 1 wherein said apparatus is capable of delivering dry medication.

4. The apparatus of claim 3, wherein user engagement and inhalation at said mouthpiece creates an airflow stream which passes through said inflow, past said fixed resistor and past said pressure transducer and into the pulmonary tract of a patient.

5. The apparatus of claim 4, wherein pressure changes occurring in said airflow stream passing through said housing are detected by said pressure transducer, said pressure transducer creating a signal proportional to said pressure change.

6. The apparatus of claim 5, wherein said signal is received by said microprocessor, said microprocessor enabled to perform logical operations or interpretive calculations upon said signal

7. The apparatus of claim 6, wherein said microprocessor measures said signals over several breaths to calculate an average flow rate and tidal volume.

8. The apparatus of claim 7, wherein said average flow rate measured by said microprocessor initiates the generation of a medication-to-air ratio used to meter a proper dose of medication for delivery at a desired time.

9. The apparatus of claim 1, wherein said apparatus may be activated by a user through use of a closed-mouth technique.

10. The apparatus of claim 1, wherein said apparatus may be activated by a user through use of an open-mouth technique.

11. The apparatus of claim 1, wherein said apparatus may be manually activated.

12. The apparatus of claim 1, wherein said microprocessor controls the release of a unit dose of a medicament delivered from said canister.

13. The apparatus of claim 12, wherein said dose is delivered in increments over several breaths until the entire dose is administered.

14. The apparatus of claim 12, wherein said dose is delivered at different stages of an inspiratory cycle depending upon the desired site of treatment.

15. The apparatus of claim 8, wherein said microprocessor performs different logical operations or interpretive calculations based on the type of medication contained in said canister.

16. The apparatus of claim 8, wherein said microprocessor performs different logical operations or interpretive calculations based on the physical characteristics, age or other medically relevant data of a user to determine the proper dose of a medication.

17. The apparatus of claim 8, wherein said microprocessor draws power from a battery on said housing.

18. The apparatus of claim 8, wherein said microprocessor draws power from a battery on said canister.

19. The apparatus of claim 1, further comprising a usage recorder cooperating with said housing, said usage recorder enabled to indicate to a user the total doses remaining and when to administer subsequent doses.

20. The apparatus of claim 19, wherein said usage recorder employs at least one auditory, visual or vibratory indicator.

21. The apparatus of claim 19, wherein said usage recorder draws power from a battery on said housing.

22. The apparatus of claim 19, wherein said usage recorder draws power from a battery on the canister.

23. A breath-activated, microprocessor controlled apparatus for pulmonary delivering of air-mixed medication comprising:
a). a housing having a proximal end comprising an inflow with a fixed resistor and a distal end comprising a mouthpiece, wherein said mouthpiece is in communication with said inflow to permit air to flow through said housing;
b). a pressure transducer for measuring pressure changes in air passing through said housing, said pressure transducer enabled to generate a signal in response to said pressure change;
c). a programmable microprocessor responsive to signals generated from said pressure transducer;
d). a removable canister containing a drug formulation;
e) electrical circuitry, wherein said circuitry communicates with said pressure transducer, said microprocessor, and said canister; and
f). an actuator enabled to cause the release of a dose of a drug formulation from said canister.

23. The apparatus of claim 22, further comprising a gate in communication with said housing for allowing air to be mixed with medication dispersed from said canister.

24. The apparatus of claim 23 where said gate can be manually or electronically adjusted to change the resistance to air passing through the device.

25. The apparatus of claim 22, wherein said apparatus is handheld.

26. The apparatus of claim 22, wherein said microprocessor may be programmed to recognize the contents of a canister containing a drug formulation, and calculate the appropriate dose of said drug formulation.

27. The apparatus of claim 22, wherein said microprocessor may be programmed to calculate a dose of a drug formulation based on a patient's physical characteristics, age or other medically relevant factor.

28. The apparatus of claim 22, wherein said microprocessor may be programmed by a user or physician at home, in a physicians office or over the internet.

29. A method of increasing the efficiency of intrapulmonary delivery of a medicament using a breath-activated, microprocessor controlled apparatus comprising:
a). placing the proximal end of said apparatus, into the mouth; and
b). breathing normally over several breaths to create an air stream traveling from the proximal end of apparatus, through the main body, out the distal end and into the patient; and
c) inhaling air-mixed medication released from a medicine canister communicating with said apparatus, over several inspiratory cycles until the entire dose is administered.

30. The method of claim 29, wherein said pressure changes in said air stream passing through said housing are detected by a pressure transducer communicating with said housing.

31. The method of claim 30, wherein in said pressure transducer generates a signal in response to said pressure changes.

32. The method of claim 31, wherein said signal is received by a microprocessor, said microprocessor enabled to perform logical operations or interpretive calculations upon said signal to generate a proper dose of medicine.

33. The method of claim 32, wherein said dose is released from a replaceable aerosol medication canister in response to a signal generated from said microprocessor.

34. The method of claim 33, wherein said dose is administered incrementally over several breaths until the entire dose is received.

35. The method of claim 34, wherein said incremental administration increases the degree of evaporation of said medicine, said evaporation decreasing the particle size of the drug to a respirable range while increasing the amount of medicine reaching the pulmonary or systemic circulation.

36. The method of claim 35, wherein said evaporation decreases the degree of oropharyngeal deposition, accidental swallowing, or other waste of said medicine.

37. The method of claim 29, wherein said microprocessor monitors patient breathing over several breaths to calculate an average flow rate and tidal volume, and meters an appropriate dose over several additional breaths to insure maximum efficiency of drug delivery.

38. The method of claim 37, wherein said microprocessor evaluates the parameters of each breath factoring in the correct timing of the inspiratory cycle, correct flow rate and how much drug remains to be given to determine whether the medicament was appropriately given.

39. The method of claim 39, wherein said microprocessor provides auditory, visual or vibratory feedback to a patient to indicate whether medicament was adequately delivered.

40. The method of claim 37, wherein said medicine is delivered to varying locations in the brocho-pulmonary tree depending upon the flow rate generated by a patient, the type of medicine used, or both.
